(19) United States
(12) Patent Application Publication
(10) Pub. No.: US 2009/0081689 A1
(43) Pub. Date: Mar. 26, 2009

(54) Reagents and methods to enrich rare cells from body fluids

(76) Inventors: Douglas Yamanishi, San Diego, CA (US); Roopa Srinivasan, San Marcos, CA (US)

Correspondence Address: DAVID R PRESTON & ASSOCIATES APC, 5850 OBERLIN DRIVE, SUITE 300, SAN DIEGO, CA, 92121, US

(21) Appl. No.: 12/284,655

(22) Filed: Sep. 24, 2008

Related U.S. Application Data
(60) Provisional application No. 60/995,201, filed on Sep. 25, 2007.

(51) Int. Cl. C12Q 1/68; C12N 5/06; G01N 33/567

(52) U.S. Cl. 435/6; 435/374; 435/7.21

(57) ABSTRACT

The present invention relates to compositions and methods for separating cells. The present invention utilizes a combination of techniques to deplete non-nucleated and nucleated cells in a biological sample. The use of this invention assists in reducing the complexity of a biological sample such as peripheral blood, and help in the diagnosis and prognosis of many conditions. The invention includes solutions and methods to selectively deplete red and white blood cells from a blood sample. The preferred sample is blood, effusion, or aspirate samples containing one or more cell types that can be enriched from such a sample. The present invention provides methods for identifying target cells, nucleic acids or chromosome quantification.

### CROSS REFERENCE TO RELATED APPLICATIONS

The present invention claims benefit of priority to U.S. provisional patent application Ser. No. 60/995,201, filed on Sep. 25, 2007, the entire contents of which are incorporated herein by reference in its entirety.

### TECHNICAL FIELD

The present invention relates to the field of bioseparation, and in particular to the field of biological sample processing. The present invention also relates to a method of diagnosing genetic abnormalities using a minimally invasive approach, for the identification of chromosomal and/or DNA alterations using cells obtained from body fluids.

### BACKGROUND

Sample preparation is a necessary step for many genetic, biochemical, and biological analyses of biological and environmental samples. Sample preparation frequently requires the separation of sample components of interest from the remaining components of the sample. In some cases, sample processing is required to enrich target cells of low abundance. This would necessitate the need to remove undesirable components that could interfere with the analysis of the components of interest. The sample may need to be “debulked” to reduce its volume or the undesirable components in order to enrich the components of interest. This situation is true for biological samples, (such as ascites fluid, lymph fluid, blood, or bone marrow aspirates) that can harvested from a body in large amounts but contain minute percentages of target cells (such as virus-infected cells, tumor cells, T-cells, fetal cells, stem cells, or bacteria-infected cells).

Blood samples provide special challenges for sample preparation and analysis. Blood samples are easily obtained from subjects and a test material from blood can provide a wealth of information (metabolic, diagnostics, prognostic, and genetic). This test material is often found in very small numbers in blood and the great abundance of non-nucleated red blood cells (RBCs) and white blood cells (WBCs) can be an impediment to these tests.

Commonly known protocols to deplete RBCs utilize solutions to chemically induce RBC aggregation. Such solutions would include dextran or hepastarch to induce RBC aggregation (Sewchand, L. S. and Canham P B “Modes of rouleaux formation of human red blood cells in polyvinylpyrrolidone and dextran solutions. Can. J. Physiol. Pharmacol 57(11): 1213-1222, 1979). A commonly known way is to lyse RBCs using chemical solutions such as acetic acid or ammonium chloride solutions. A third way is to layer blood onto a density gradient (e.g. U.S. Pat. No. 5,437,987 to Nelson Teng et al) to remove RBCs and WBCs.

Commonly known protocols to deplete WBCs utilize magnetic beads coated with an antibody(s) specific for WBC antigens. This technology uses a magnetic field to capture the WBCs and is a commonly accepted protocol. Another technology utilizes antibodies to WBC antigens linked to an antibody that binds to an antigen on RBCs (e.g. glycophorin A). The linking of WBCs to RBCs (using an antibody to antibody link) in combination with a density gradient can separate RBCs and WBCs attached to RBCs from the non-attached WBCs.

Exfoliated cells in body fluids (e.g. sputum, urine, or even ascetic fluid or other effusions) present a significant opportunity for the detection of precancerous lesions and therefore eradication or treatment of cancer at early stages of neoplastic development. Biomarker studies and the use of biomarkers for clinical practice require a relatively pure exfoliated cell population enriched from body fluids comprising not only the exfoliated cells but also normal cells, bacteria, body fluids, body proteins and other cell debris. There is an immediate need for developing an effective enrichment method for enriching and isolating exfoliated abnormal cells from body fluids.

Upon enrichment of the target cells, these cells can be evaluated. One target cell which can utilize this invention would be the enrichment of fetal cells from peripheral blood for prenatal diagnosis. Prenatal diagnosis involves the identification of fetal malformations or genetic abnormalities in a human fetus.

Current technology to detect fetal abnormalities include: ultrasound scans, serum protein analysis, and genetic tests. Ultrasound scans can usually detect structural malformations involving the neural tube, heart, kidney, limbs, and other organs. Serum protein levels can be used to potentially detect the possibility of fetuses with chromosome abnormalities but lack sensitivity and specificity. On the other hand, invasive samplings such as chorionic villus sampling or amniocentesis are used to identify fetuses with chromosome abnormalities. These invasive procedures, while very accurate for detection of chromosome abnormalities, have issues including a 0.5-2% procedure related risk of miscarriage and may be associated with an increased risk of fetal structural abnormalities.

Several approaches have been developed to improve the detection of fetal abnormalities using non- or minimal invasive procedures. These approaches used body fluids from pregnant women to isolate fetal cells. The fetal cells that could be enriched include: leukocytes, trophoblasts, and nucleated red blood cells. Fetal leukocytes have the issue that they may persist in peripheral blood as long as 27 years after child birth (Bianchi D W et al. Proc Natl Acad Sci 93(2): 705-708, 1996). Fetal trophoblasts enriched from blood can have the issue of multinucleated morphology, which are difficult for genetic analysis using interphase fluorescent in situ hybridization. Nucleated fetal red blood cells have a relatively short half-life of 90 days and have been the target of many studies (for review, see Bianchi D W et al. Prenat Diagn 22(7): 609-615, 2002).

Several procedures have been implemented to isolate fetal cells ranging from density gradients to magnetic separation (for review, see Bianchi D W et al. Prenat Diagn 22(7): 609-615, 2002 and Yamanishi D T et al Exp Rev Mol Diagn 2(4): 303-311, 2002). Centrifugation with density gradients or red blood cell lysis, FACS, or antibodies combined with magnetic beads has generally resulted in the enrichment of maternal cells with inconsistent and non-robust recovery of fetal cells. The combinations of the high cost and technical issues have inhibited the utilization of minimally invasive procedures for fetal cell isolation in clinical practice.

For handling biological fluids, there is also a need to provide methods of sample preparation that are efficient and automatable. Current approaches for enriching cells from body fluids are through density based separation, paramagnetic capture, centrifugation and membrane-based filtration. While these techniques are simple and straightforward, they suffer from a number of limitations, including: inadequate recovery of rare cells, low sensitivity of rare cells, difficulty in either handling complex mixtures or large volumes, and require labor intensive procedures. The present invention provides these and other benefits.

### SUMMARY

The present invention recognizes that diagnosis, prognosis and treatment of several medical conditions can depend on the enrichment of rare cells from a complex biological specimen. This present invention relates to the fields of cell separation and cell identification. Particularly, this invention provides methods and compositions for the enrichment and identification of individual cells in samples from body fluids, particularly from peripheral blood.

The enrichment of fetal cells from maternal blood samples can aid in the detection of fetal abnormalities. In addition, the enrichment of malignant cells from patients with cancer can aid in the detection of circulating cancer cells which will aid in the diagnosis and prognosis of the disease stage of the patient. Another aspect of the invention also helps in the development of therapeutic modalities for patients. Another example is to enrich stem or progenitor cells for therapeutic modalities.

The present invention describes reagents and methods for enriching rare cells of a blood sample. In one aspect, a red blood cell sedimenting solution of the present invention comprises a reagent to induce red blood cell (RBC) aggregation and at least one specific binding member that can specifically bind red blood cells (RBCs). In preferred embodiments, a combined solution includes a specific binding member that specifically binds white blood cells (WBCS) that is bound to beads or particles that can bind to other WBCS. In some preferred embodiments, a combined solution for enriching rare cells of a blood sample comprises of a RBC aggregation reagent, at least one specific binding member that can selectively bind RBCS, at least one specific binding member that specifically binds undesirable components of a sample and at least one specific binding reagent that can induce white blood cells to aggregate. The present invention includes methods to identify cells or nucleic acids or to quantify nucleic acid copies.

### BRIEF DESCRIPTION OF THE DRAWINGS

FIG. 1 is a representation of a few steps in a cell separation method of the present invention. A is the initial sample. B is the sample after addition of the reagents and mixing of the solution. There is a mixture of cell aggregates and single cells. C is the mixture after the settling step.

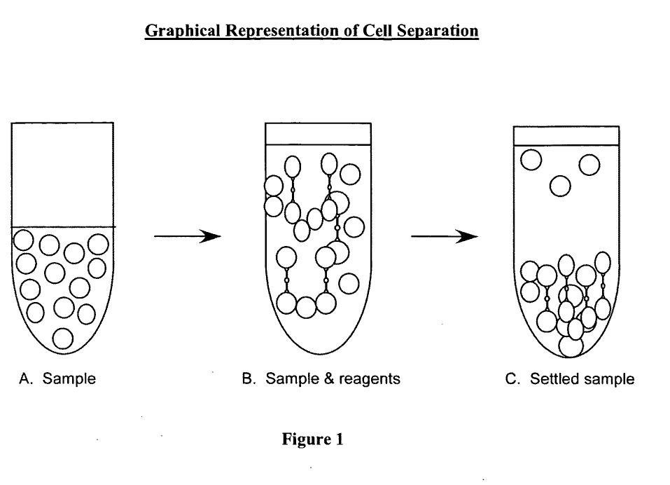

FIG. 2 is a representation of different examples of cell aggregates that could form during the method. Single cells are represented as large circles. Antibodies and beads are represented as lines and small circles.

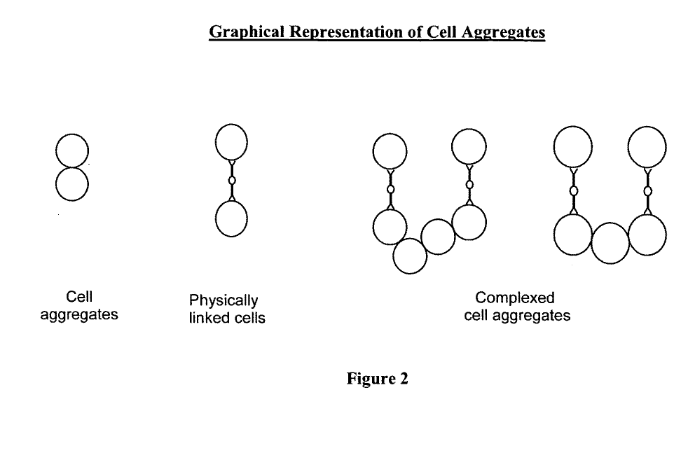

FIG. 3 is a representation of the method to reduce the complexity of nucleic acids from two samples and determine copy number differences. Nucleic acids are shown as straight lines. Paired lines are double stranded and single lines are single stranded. Primers are shown as boxes. A part is of the primer is shaded that contains a non-homologous regions with a high melting temperature (Tm).

### DETAILED DESCRIPTION OF THE INVENTION

### A. Definitions

Unless defined otherwise, all technical and scientific terms used herein have the same meaning as commonly understood by one of ordinary skill in the art to which this invention belongs. Generally, the nomenclature used herein and the manufacture procedures for devices and components described below are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings.

A “component” of a sample or “sample component” is any constituent of a sample, and can be an ion, molecule, compound, molecular complex, organelle, virus, bacteria, cell, aggregate, or particle of any type, including colloids, aggregates, particulates, crystals, minerals, etc. A component of a sample can be soluble or insoluble in the sample media or a provided sample buffer or sample solution. A component of a sample can be in gaseous, liquid, or solid form. A component of a sample may be a moiety or may not be a moiety.

A “moiety” or “moiety of interest” is any entity whose manipulation is desirable. A moiety can be a solid, including a suspended solid, or can be in soluble form. A moiety can be a molecule. Molecules that can be manipulated include, but are not limited to, inorganic molecules, including ions and inorganic compounds, or can be organic molecules, including amino acids, peptides, proteins, glycoproteins, lipoproteins, glycolipoproteins, lipids, fats, sterols, sugars, carbohydrates, nucleic acid molecules, small organic molecules, or complex organic molecules. A moiety can also be a molecular complex, can be an organelle, can be one or more cells, including prokaryotic and eukaryotic cells, or can be one or more etiological agents, including viruses, bacteria, parasites, or prions, or portions thereof. A moiety can be a crystal, mineral, colloid, fragment, mycelle, micelle, droplet, bubble, or the like, and can comprise one or more inorganic materials such as polymeric materials, metals, minerals, glass, ceramics, and the like. Moieties can also be aggregates of molecules, complexes, cells, organelles, viruses, bacteria, etiological agents, crystals, colloids, or fragments. Cells can be any cells, including prokaryotic and eukaryotic cells. Eukaryotic cells can be of any type. Of particular interest are cells such as, but not limited to, white blood cells, malignant cells, stem cells, progenitor cells, fetal cells, and cells infected with an etiological agent, and bacterial cells. Moieties can also be artificial particles such polystyrene microbeads made of polystyrene or other polymer compositions, magnetic microbeads, particles of polystyrene or other polymer compositions, microspheres of polystyrene or other polymer compositions, and carbon microbeads.

“Binding partner” refers to any substances that both bind to the moieties with desired affinity or specificity and are manipulatable with the desired physical force(s). Non-limiting examples of the binding partners include cells, cellular organelles, viruses, bacteria, microparticles or an aggregate or complex thereof, or an aggregate or complex of molecules.

A “microparticle” or “particle” is a structure of any shape and of any composition that is manipulatable by desired physical force(s). The microparticles used in the methods could have a dimension from about 0.01 micron to about ten centimeters. Preferably, the microparticles used in the methods can be comprised of any suitable material, such as glass or ceramics, and/or one or more polymers, such as, for example, nylon, polytetrafluoroethylene, polystyrene, polyacrylamide, sepharose, agarose, cellulose, cellulose derivatives, or dextran, and/or can comprise metals. Examples of microparticles include, but are not limited to, plastic particles, ceramic particles, carbon particles, polystyrene microbeads, glass beads, magnetic beads, hollow glass spheres, metal particles, particles of complex compositions, microfabricated or micromachined particles, etc.

“Coupled” means bound. For example, a moiety can be coupled to a microparticle by specific or nonspecific binding. As disclosed herein, the binding can be covalent or noncovalent, reversible or irreversible. As used herein, “the moiety to be manipulated is substantially coupled onto surface of the binding partner” means that a percentage of the moiety to be manipulated is coupled onto surface of the binding partner and can be manipulated by a suitable physical force via manipulation of the binding partner. Ordinarily, at least 0.1% of the moiety to be manipulated is coupled onto surface of the binding partner. Preferably, at least 1%, 5%, 10%, 20%, 30%, 40%, 50%, 60%, 70%, 80% or 90% of the moiety to be manipulated is coupled onto surface of the binding partner. As used herein, “the moiety to be manipulated is completely coupled onto surface of the binding partner” means that at least 90% of the moiety to be manipulated is coupled onto surface of the binding partner. Preferably, at least 91%, 92%, 93%, 94%, 95%, 96%, 97%, 98%, 99% or 100% of the moiety to be manipulated is coupled onto surface of the binding partner.

A “specific binding member” is one of two different molecules having an area on the surface or in a cavity which specifically binds to and is thereby defined as complementary with a particular spatial and chemical organization of the other molecule. A specific binding member can be a member of an immunological pair such as antigen-antibody, antibody-antibody, protein-protein, lectin-antigen, biotin-avidin, biotin-streptavidin, biotin-neutravidin, ligand-receptor, nucleic acid duplexes, Ig-protein A, Ig-protein G, Ig-protein L, Ig-protein A/G, DNA-DNA, DNA-RNA, RNA-RNA, or the like.

An “antibody” is an immunoglobulin molecule, and can be, as nonlimiting example, an IgG, an IgM, an IgE, an IgA or other type of immunoglobulin molecule. As used herein, “antibody” also refers to a portion of an antibody molecule that retains the binding specificity of the antibody from which it is derived (for example, single chain antibodies or Fab fragments).

A “fluid sample” is any fluid from which components are to be separated or analyzed. A sample can be from any source, such as an organism, group of organisms from the same or different species, from the environment, such as from a body of water or from the soil, or from a food source or an industrial source. A sample can be an unprocessed or a processed sample. A sample can be a gas, a liquid, or a semi-solid, and can be a solution or a suspension. A sample can be an extract, for example a liquid extract of a soil or food sample, an extract of a throat or genital swab, or an extract of a fecal sample, or a wash of an internal area of the body.

A “blood sample” as used herein can refer to a processed or unprocessed blood sample, i.e., it can be a centrifuged, filtered, extracted, or otherwise treated blood sample, including a blood sample to which one or more reagents such as, but not limited to, anticoagulants or stabilizers have been added. An example of blood sample is a buffy coat that is obtained by processing human blood for enriching white blood cells. Another example of a blood sample is a blood sample that has been “washed” to remove serum components by centrifuging the sample to pellet cells, removing the serum supernatant, and resuspending the cells in a solution or buffer. Other blood samples include cord blood samples, bone marrow aspirates, blood from organ, internal blood or peripheral blood. A blood sample can be of any volume, and can be from any subject such as an animal or human. The preferred subject is a primate, preferably homo sapiens.

A “rare cell” is a cell that is either 1) of a cell type that is less than 1% of the total nucleated cell population in a fluid sample, 2) of a cell type that is less than 1% of the total cell population in sample, or 3) of a cell type that is present at less than one million cells per milliliter of fluid sample. A “rare cell of interest” is a cell whose enrichment is desirable.

A “white blood cell” or “WBC” is a leukocyte, or a cell of the hematopoietic lineage that is not a reticulocyte or red blood cell or nucleated red blood cell or platelet and that can be found in the blood of an animal or human. Leukocytes can include nature killer cells (“NK cells”) and lymphocytes, such as B lymphocytes (“B cells”) or T lymphocytes (“T cells”). Leukocytes can also include phagocytic cells, such as monocytes, macrophages, and granulocytes, including basophils, eosinophils and neutrophils. Leukocytes can also comprise mast cells.

A “red blood cell” or “RBC” is an erythrocyte. Unless designated a “nucleated red blood cell” (“nRBC”) or “fetal nucleated red blood cell”, as used herein, “red blood cell” is used to mean a non-nucleated red blood cell.

“Neoplastic cells” refers to abnormal cells that have uncontrolled cellular proliferation and can continue to grow after the stimuli that induced the new growth has been withdrawn. Neoplastic cells tend to show partial or complete lack of structural organization and functional coordination with the normal tissue, and may be benign or malignant.

A “malignant cell” is a cell having the property of locally invasive and destructive growth and metastasis. Examples of “malignant cells” include, but not limited to, leukemia cells, lymphoma cells, cancer cells of solid tumors, metastatic solid tumor cells (such as but not limited to breast cancer cells, prostate cancer cells, lung cancer cells, colon cancer cells) in various body fluids including blood, bone marrow, ascitic fluids, stool, urine, bronchial washes, etc.

A “cancerous cell” is a cell that exhibits deregulated growth and, in most cases, has lost at least one of its differentiated properties, such as, but not limited to, characteristic morphology, non-migratory behavior, cell-cell interaction and cell-signaling behavior, protein expression and secretion pattern, etc.

A “stem cell” is an undifferentiated cell that can give rise, through one or more cell division cycles, to at least one differentiated cell type.

A “progenitor cell” is a committed but undifferentiated cell that can give rise, through one or more cell division cycles, to at least one differentiated cell type. Typically, a stem cell gives rise to a progenitor cell through one or more cell divisions in response to a particular stimulus or set of stimuli, and a progenitor gives rise to one or more differentiated cell types in response to a particular stimulus or set of stimuli.

An “etiological agent” refers to any etiological agent, such as a bacteria, fungus, protozoan, virus, parasite or prion that can infect a subject. An etiological agent can cause symptoms or a disease state in the subject it infects. A human etiological agent is an etiological agent that can infect a human subject. Human etiological agents may be specific for humans, such as a specific human etiological agent, or may infect a variety of species, such as a promiscuous human etiological agent.

“Subject” refers to any organism, such as an animal or a primate, preferably homo sapiens. An animal can include any animal, such as a feral animal, a companion animal such as a dog or cat, an agricultural animal such as a pig or a cow, or a pleasure animal such as a horse.

“Processing” refers to the preparation of a sample for analysis, and can comprise one or multiple steps or tasks. Generally a processing task serves to separate components of a sample, concentrate components of a sample, at least partially purify components of a sample, or structurally alter components of a sample (for example, by lysis or denaturation).

As used herein, “isolating” means separating a desirable sample component from other nondesirable components of a sample, such that preferably, at least 15%, more preferably at least 30%, even more preferably at least 50%, and further preferably, at least 80% of the desirable sample components present in the original sample are retained, and preferably at least 50%, more preferably at least 80%, even more preferably, at least 95%, and yet more preferably, at least 99%, of at least one nondesirable component of the original component is removed, from the final preparation.

“Selectively binds” means that a specific binding member used in the methods of the present invention to remove one or more undesirable sample components does not appreciably bind to rare cells of interest of the sample. The term “does not appreciably bind” means that not more than 30%, preferably not more than 20%, more preferably not more than 10%, and yet more preferably not more than 1.0% of one or more rare cells of interest are bound by the specific binding member used to remove non-RBC undesirable components from the fluid sample. In many cases, the undesirable components of a blood sample will be white blood cells. In preferred embodiments of the present invention, a combined solution of the present invention can be used for sedimenting red blood cells and selectively removing white blood cells from a blood sample.

“Enrich” means increase the concentration of a sample component of a sample relative to other sample components (which can be the result of reducing the concentration of other sample components), or increase the concentration of a sample component. For example, as used herein, “enriching” fetal nucleated red blood cells from a blood sample means increasing the proportion of fetal nucleated red blood cells to all cells in the blood sample, enriching cancer cells of a blood sample can mean increasing the concentration of cancer cells in the sample (for example, by reducing the sample volume) or reducing the concentration of other cellular components of the blood sample, and “enriching” cancer cells in a urine sample can mean increasing the cancer cell concentration in the sample.

“Separation” is a process in which one or more components of a sample are spatially separated from one or more other components of a sample. A separation can be performed such that one or more sample components of interest is translocated to or retained in one or more areas of a separation apparatus and at least some of the remaining components are translocated away from the area or areas where the one or more sample components of interest are translocated to and/or retained in, or in which one or more sample components is retained in one or more areas and at least some or the remaining components are removed from the area or areas. Alternatively, one or more components of a sample can be translocated to and/or retained in one or more areas and one or more sample components can be removed from the area or areas. Separations can be achieved through, for example, filtration, or the use of physical, chemical, electrical, or magnetic forces. Nonlimiting examples of forces that can be used in separations are gravity, centrifugal force, mass flow, magnetic, dielectrophoretic forces, traveling-wave, and electromagnetic forces.

“Separating a sample component from a (fluid) sample” means separating a sample component from other components of the original sample, or from components of the sample that are remaining after one or more processing steps.

“Removing a sample component from a (fluid) sample” means removing a sample component from other components of the original sample, or from components of the sample that are remaining after one or more processing steps.

“Capture” is a type of separation in which one or more moieties or sample components is retained in or on one or more areas of a surface, chamber, chip, tube, or any vessel that contains a sample, where the remainder of the sample can be removed from that area.

An “assay” is a test performed on a sample or a component of a sample. An assay can test for the presence of a component, the amount or concentration of a component, the composition of a component, the activity of a component, etc. Assays that can be performed in conjunction with the compositions and methods of the present invention include, but not limited to, immunocytochemical assays, interphase FISH (fluorescence in situ hybridization), karyotyping, immunological assays, biochemical assays, binding assays, cellular assays, genetic assays, gene expression assays and protein expression assays.

A “binding assay” is an assay that tests for the presence or concentration of an entity by detecting binding of the entity to a specific binding member, or that tests the ability of an entity to bind another entity, or tests the binding affinity of one entity for another entity. An entity can be an organic or inorganic molecule, a molecular complex that comprises, organic, inorganic, or a combination of organic and inorganic compounds, an organelle, a virus, or a cell. Binding assays can use detectable labels or signal generating systems that give rise to detectable signals in the presence of the bound entity. Standard binding assays include those that rely on nucleic acid hybridization to detect specific nucleic acid sequences, those that rely on antibody binding to entities, and those that rely on ligands binding to receptors.

A “biochemical assay” is an assay that tests for the presence, concentration, or activity of one or more components of a sample.

A “cellular assay” is an assay that tests for a cellular process, such as, but not limited to, a metabolic activity, a catabolic activity, an ion channel activity, an intracellular signaling activity, a receptor-linked signaling activity, a transcriptional activity, a translational activity, or a secretory activity.

A “genetic assay” is an assay that tests for the presence or sequence of a genetic element, where a genetic element can be any segment of a DNA or RNA molecule, including, but not limited to, a gene, a repetitive element, a transposable element, a regulatory element, a telomere, a centromere, or DNA or RNA of unknown function. As nonlimiting examples, genetic assays can be gene expression assays, PCR assays, karyotyping, or FISH. Genetic assays can use nucleic acid hybridization techniques, can comprise nucleic acid sequencing reactions, or can use one or more enzymes such as polymerases, as, for example a genetic assay based on PCR. A genetic assay can use one or more detectable labels, such as, but not limited to, fluorochromes, radioisotopes, or signal generating systems.

“FISH” or “fluorescence in situ hybridization” is an assay wherein a genetic marker can be localized to a chromosome by hybridization. Typically, to perform FISH, a nucleic acid probe that is fluorescently labeled is hybridized to interphase chromosomes that are prepared on a slide. The presence and location of a hybridizing probe can be visualized by fluorescence microscopy. The probe can also include an enzyme and be used in conjunction with a fluorescent enzyme substrate.

“Karyotyping” refers to the analysis of chromosomes that includes the presence and number of chromosomes of each type (for example, preferably each of the 24 chromosomes of the human haplotype (chromosomes 1-22, X, and Y)), and the presence of morphological abnormalities in the chromosomes, such as, for example, translocations or deletions. Karyotyping typically involves performing a chromosome spread of a cell in metaphase. The chromosomes can then be visualized using, for example, but not limited to, stains or genetic probes to distinguish the specific chromosomes.

A “gene expression assay” (or “gene expression profiling assay”) is an assay that tests for the presence or quantity of one or more gene expression products, i.e. messenger RNAs. the one or more types of mRNAs can be assayed simultaneously on cells of the interest from a sample. For different applications, the number and/or the types of mRNA molecules to be assayed in the gene expression assays may be different.

A “protein expression assay” (or “protein expression profiling assay”) is an assay that tests for the presence or quantity of one or more proteins. One or more types of protein can be assayed simultaneously on the cells of the interest from a sample. For different applications, the number and/or the types of protein molecules to be assayed in the protein expression assays may be different.

“Histological examination” refers to the examination of cells using histochemical or stains or specific binding members (generally coupled to detectable labels) that can determine the type of cell, the expression of particular markers by the cell, or can reveal structural features of the cell (such as the nucleus, cytoskeleton, etc.) or the state or function of a cell. In general, cells can be prepared on slides and “stained” using dyes or specific binding members directly or indirectly bound to detectable labels, for histological examination. Examples of dyes that can be used in histological examination are nuclear stains, such as Hoechst stains, or cell viability stains, such as Trypan blue, or cellular structure stains such as Wright or Giemsa, enzyme activity benzidine for HRP to form visible precipitate. Examples of specific binding members that can be used in histological examination of fetal red blood cells are antibodies that specifically recognize fetal or embryonic hemoglobin.

Other technical terms used herein have their ordinary meaning in the art that they are used, as exemplified by a variety of technical dictionaries.

B. Reagents and Methods to Enrich Rare Cells from Body Fluids

The present invention recognizes that to analyze complex fluids, such as biological fluid samples, an enrichment for the target cells or bioanalyte may be required which will remove components in the original sample. The enrichment may be required since component or components may interfere with sample analysis. Sample analysis of peripheral blood samples may also have problems when the target is a rare cell type, such as fetal cells, cancer cells, stem cells or progenitor cells. In processing such samples, it is often necessary to both “debulk” the sample, by reducing the volume to a manageable level, and to enrich the population of rare cells that are the target for analysis. Procedures for the processing of fluid samples are often time consuming and inefficient. The present invention provides efficient methods and automated systems for the enrichment of rare cells from fluid samples.

As a non-limiting introduction to the breath of the present invention, the present invention includes several general and useful aspects, including:
1) a method of enriching rare cells from a body fluid.
2) solutions for the selective sedimentation of red or white blood cells comprising at least one cell aggregating reagent and at least one specific binding member that selectively binds red or white cells.
3) methods of using selective RBC sedimentation solutions and WBC sedimentation solutions for enriching rare cells in a biological fluid.
   These aspects of the invention, as well as others described herein, can be achieved by using the methods, articles of manufacture and compositions of matter described herein. To gain a full appreciation of the scope of the present invention, it will be further recognized that various aspects of the invention can be combined to make desirable embodiments of the invention.

### Sample

A sample can be any fluid sample, such as an environmental sample, including air samples, water samples, food samples, and biological samples, including suspensions, extracts, or leachates of environmental or biological samples. Biological samples can be blood, bone marrow sample, effusion of any type, ascites fluid, pelvic wash fluid, pleural fluid, spinal fluid, lymph, serum, mucus, sputum, saliva, urine, semen, ocular fluid, extracts of nasal, throat or genital swabs, cell suspension from digested tissue, washes or fluids from organs or tissue digests, or extracts of fecal material. Biological samples can also be samples of organs or tissues, including tumors, such as fine needle aspirates or samples from perfusions of organs or tissues.

Biological samples can be samples of cell cultures, including both primary cultures and cell lines. The volume of a sample can be very small, such as in the microliter range, and may require dilution, or a sample can be very large, such as up to liters for ascites fluid. A preferred sample is a blood sample. A blood sample can be any blood sample, recently taken from a subject, taken from storage, or removed from a source external to a subject, such as clothing, upholstery, tools, etc. A blood sample can therefore be an extract obtained, for example, by soaking an article containing blood in a buffer or solution. A blood sample can be unprocessed or partially processed, for example, a blood sample that has been dialyzed, centrifuged, filtered, had reagents added to it, etc. A blood sample can be of any volume. For example, a blood sample can be less than five microliters, or more than 5 liters, depending on the application.

The rare cells to be enriched from a sample can be of any cell type present at less than one million cells per milliliter of fluid sample, constitute less than 1% of the total nucleated cell population in a fluid sample or constitute less than 1% of the total cell population in a fluid sample. Rare cells can be, for example, bacterial cells, fungal cells, parasitic cells, infected cells (e.g. parasites, bacteria, or viruses), or eukaryotic cells such as but not limited to fibroblasts or blood cells. Rare blood cells can be RBCs (for example, if the sample is an extract or leachate containing less than than one million cells per milliliter RBCs), subpopulations of blood cells and blood cell types, such as WBCs, or subtypes of WBCs (for example, T cells or macrophages), or can be nucleated red blood cells, including fetal nucleated red blood cells. Rare cells can be stem cells or progenitor cells of any type. Rare cells can be tumor cells, including cancer cells, neoplastic cells or malignant cells. Rare cells of a blood sample can be non-hematopoietic cells, such as but not limited to epithelial cells.

### Debulking

In preferred aspects of the present invention in which the fluid sample is a blood sample, a majority of the non-nucleated red blood cells (RBCs) that make up more than 90% of the cellular components of a blood sample can be removed during a debulking step.

A debulking step can be, as nonlimiting examples, a selective sedimentation step, a selective lysis step, a concentration step, a centrifugation step, or a filtration step. Preferred debulking steps are those that reduce the volume of a fluid sample and at the same time allow the technician to select portions of the centrifuged, filtered, or selectively sedimented product that retain desirable components and do not retain at least a portion of some undesirable components.

Centrifugation can reduce the volume of a sample by pelleting insoluble components of a sample, or can separate components on the basis of density, and can make use of density gradients that can also separate components of a sample, such as different cell types. The one or more mononuclear layers are separated from the other layers to obtain a fraction that is enriched in fetal nucleated red blood cells.

Another method for debulking of blood is through selective sedimentation of erythrocytes (red blood cells) by using certain reagents. Preferably, such agglutination and sedimentation of erythrocytes does not affect the cells of interest in the blood samples, and the loss of the target cells (i.e. the cells of interest) is as small as possible. For example, hepastarch is a cell separation reagent, which can be used for such purposes. HepaSep (StemCell Technologies, Vancouver, Canada) can significantly (99%) remove RBCs according to product literature.

Another method for debulking blood sample is the use of hypotonic solutions. By treating blood samples with hypotonic solutions, red blood cells can be selectively lysed, or red blood cells can be altered significantly so that they become readily separable from white blood cells and other nucleated cells. Some solutions that selectively lyse red blood cells are described in U.S. patent application Ser. No. 09/973,629 incorporated by reference in their entireties.

### Cell Separation

Previous work has developed antibodies that are linked together to induce RBCs linked to RBCs and WBCs linked to RBCs (StemCell Technologies, Vancouver, Canada). Current technology crosslinks antibodies (antibody to white blood cell linked via an antibody or physical surface to another antibody to red blood cell or a linkage of antibody to red blood cell to antibody to red blood cell). The limitation of these technologies is that the bound cell needs to bind to red blood cells or the bound cell is not depleted. The reagents will induce clumps of RBCs linked to RBCs (RBCs/RBCs) and RBCs linked to WBCs (RBCs/WBCs), which results in a complex mixture of RBC and WBC linkages that utilize the RBC differences to deplete WBCs. The RBC/WBC and RBC/RBC clumps are depleted using a density gradient. The limitation is the need for a complex mixture of linked antibodies to link RBCs to RBCs and RBCs to WBCs and the need for density gradients.

Current technology utilizes antibody coated bead(s) to bind to a target cell to be depleted (for example, companies that sell magnetic beads include Invitrogen, Carlsbad, Calif.: Miltenyi Biotec, Auburn, Calif.; and Immunicon, Huntingdon Valley, Pa.). In this technology, multiple small paramagnetic beads (≦1 micron) are used to deplete a single cell. The use of large paramagnetic beads (˜5 micron) removes a maximum of 1 cell per bead. However, there are a few drawbacks to this approach. Firstly, this method requires a large amount of magnetic beads (5-40 beads per background cells) to result in a depletion of cells by about of 1-4 logs. Secondly, there is the potential of non-specific binding of magnetic beads to cells, which can result in the loss of target cells. Thirdly, this technology requires a magnetic field to deplete magnetic bead bound cells. A limiting factor in this scenario is the number of magnetic beads that are required to isolate a cell should the antibody bind to the cell.

### C. Cell Enrichment

The first aspect of the present invention provides the method for enriching rare cells from a blood sample. The disclosed composition and methods can be used, for example, to prepare cells for research purposes, diagnostics or therapeutics. Methods of the invention include using the reagents on blood cell containing samples (for example, blood, cord blood, bone marrow).

The present invention is based on utilizing mobile particle (for example, bead or particle) and reagents (e.g. antibodies, proteins, and/or chemicals) that can interact to deplete unwanted or background cells. One method induces non-target cells to aggregate. The other method physically links non-target cells. The combination of both methods will enhance non-target cells sticking together and result in more efficient depletion with minimal effect on the target subpopulation.

The present invention is based on using two methods to deplete background or non-target cells. One method induces the background or non-target cells to aggregate or link cells together. There is high probability for the cell aggregates to bind to each other to form a small clump.

Since this effect is a kinetic reaction, there is the possibility that the cell clumps will dissociate.

The cell could lose the antigen (resulting in an inability of cells to bind to each other). The second method using a particle can physically link the two cells together and form a direct bond between the cells. The result of using both methods is an enhancement to either independent technology.

The present invention is based on using two mechanisms to deplete unwanted cells from body fluids. Peripheral blood, as a test fluid, for example contains red blood cells (e.g. non-nucleated and nucleated) and white blood cells (e.g. granulocytes, monocytes, T-cells, and B-cells). The present invention utilizes a combination of both mechanisms: physical crosslinking and cell aggregation to result in WBC and RBC depletion from the test sample. Cell interactions can be induced targeting a specific antigen or a general response to cell type(s).

Preferably, in the methods of the present invention, selective removal of one or more undesirable components of a fluid sample makes use of specific recognition of one or more undesirable components by one or more specific binding members. A specific binding member used to remove undesirable components of a sample can be any type of molecule or substrate that can specifically bind one or more undesirable components. Receptor ligands (either naturally occurring, modified, or synthetic), antibodies, and lectins are nonlimiting examples of specific binding members that can be used in the methods of the present invention. More than one different specific binding member can be used to capture one or more undesirable components.

Specific binding members that bind to one or more undesirable components of the present invention can be used to capture one or more undesirable components, such that one or more desirable components of the fluid sample can be removed from the area or vessel where the undesirable components are bound.

### D. Solutions for Depleting Red Blood Cells

The present invention includes one or more solutions for sedimenting red blood cells of a blood sample. Red blood cell sedimenting solutions of the present invention comprise a chemical agent that induces red blood cell aggregation and at least one specific binding member that selectively binds red blood cells. When added to a blood sample, a solution for sedimenting red blood cells (an “RBC sedimenting solution”) causes red blood cells to agglutinate and sediment, and preferably does not result in the agglutination or sedimentation of substantial numbers of rare cells of interest that may be present in a blood sample.

Preferably, an RBC sedimenting solution of the present invention induces the agglutination and sedimentation of red blood cells while allowing at least 10% of rare cells whose enrichment is desired to remain in the supernatant. More preferably, an RBC sedimenting solution of the present invention induces the agglutination and sedimentation of red blood cells while allowing at least 20% of rare cells whose enrichment is desired to remain in the supernatant, and more preferably yet, an RBC sedimenting solution of the present invention allows at least 40% of rare cells whose enrichment is desired to remain in the supernatant. In the most preferred embodiments of the present invention, greater than 50% of rare cells whose enrichment is desired can be recovered from the supernatant after sedimenting RBCs with an RBC sedimenting solution of the present invention.

### RBC Aggregation Inducing Agent

Certain chemical agents can induce red blood cell (RBC) aggregation and sedimentation. For example, dextran, hespan, pentaspan, hepastarch, ficoll, gum arabic, poyvinylpyrrolidone, other natural or synthetic polymers, nucleic acids, and even some proteins can be used to induce aggregation of red blood cells (see, for example, U.S. Pat. No. 5,482,829, herein incorporated by reference). The optimal molecular weight and concentration of a chemical agent RBC aggregation inducer for aggregating red blood cells can be determined empirically.

One reagent is based on using reagents to induce RBC aggregation. A chemical or protein (such as dextran or hepastarch) can be used to induce RBC aggregation. An agent to link cells (for example but not limited to an antibody or lectin) to RBC surface markers can be included to either induce cell aggregation or improve the stability of aggregated RBCs. The combination of the two reagents can induce RBC aggregation which may result in RBC clumps that will settle with time.

A preferred chemical RBC aggregation inducing agent for use in a sedimenting solution of the present invention is a polymer such as dextran. Preferably the molecular weight of dextran in a red blood cell sedimenting solution of the present invention is between about 2 and about 2000 kilodaltons more preferably between about 50 and about 500 kilodaltons. Some preferred embodiments of the present invention are solutions comprising dextran having a molecular weight of between 70 and 200 kilodaltons. Preferably, the concentration of dextran in a red blood cell sedimenting solution of the present invention is between about 0.1% and about 20%, more preferably between about 0.2% and about 10%, and more preferably yet between about 1% and about 6%.

Specific Binding Member that Binds RBCs

Specific binding members suitable for use in a red blood cell sedimenting solution of the present invention include, as nonlimiting examples, receptor ligands or molecules comprising receptor ligands, lectins, and antibodies that can agglutinate red blood cells. One or more specific binding members that can selectively bind RBCs can be used. By “selectively binds” is meant that a specific binding member used in an RBC sedimenting solution of the present invention to does not appreciably bind to rare cells of interest of the fluid sample. By “does not appreciably bind” is meant that not more than 30%, preferably not more than 20%, more preferably not more than 10%, and yet more preferably not more than 1.0% of one or more rare cells of interest are bound by the specific binding member that binds RBCs. In some cases, it is advantageous if a specific binding member that specifically binds red blood cells is multivalent, that is, that a single specific binding member molecule or complex can specifically bind to two or more red blood cells. Where molecules such as ligands are used as specific binding members, therefore, it can be advantageous to engineer a molecule with more than one, and preferably several, ligand moieties that can bind a receptor or other red blood cell surface-exposed molecule. The optimal concentration of such a molecule in a solution of the present invention can be tested empirically.

Antibodies, especially multivalent antibodies, such as but not limited to IgG, IgA, IgE and IgM antibodies, can be preferred for use in a sedimenting solution of the present invention. Antibodies that specifically bind red blood cells are preferably antibodies that recognize one or more cell surface epitopes on red blood cells and do not appreciably bind target cells that are present in the blood sample. Concentrations of antibodies used in a solution of the present invention can vary widely, depending at least in part on the avidity of the particular antibody, from less than 0.01 microgram per milliliter to up to one milligram per milliliter of sedimenting solution. Preferably, however, an antibody used in a solution of the present invention is present at a concentration of 200 micrograms per milliliter or less. The optimal concentration of antibody used can be dependent in part on the presence and concentration of other components of the solution, including but not limited to dextran and, optionally, other specific binding members, enhancers such as oxalate, etc. (see, for example, U.S. Pat. No. 5,482,829, herein incorporated by reference). One type of antibody that can be used in a sedimenting solution of the present invention is an antibody to glycophorin A. In one preferred embodiment of the present invention, a sedimenting solution comprises an IgM antibody to glycophorin A.

Sedimenting solutions can be made and tested for their ability to sediment red blood cells and allow target cells to be recovered from the supernatant by adding the solutions to blood cells, mixing the blood sample and sedimenting solution, and incubating the blood sample for a period of time, after which the supernatant (unsedimented portion) is examined for the presence and amount of red blood cells. The volume of sedimenting solution added to the blood sample and the time of incubation can be varied in the testing of a potential sedimenting solution. Because the present invention seeks to increase the efficiency of enriching target cells of a blood sample, incubation times of less than an hour are preferred. Preferably, a red blood cell sedimenting solution of the present invention removes at least 90% of the red blood cells of a sample, more preferably, at least 95% of the red blood cells of a sample, and more preferably yet, at least 99% of the red blood cells of a sample after a mixing period of 30-60 minutes followed by a settling time of 30-60 minutes.

A red blood cell sedimenting solution of the present invention can also include other components, such as, but not limited to, salts, buffering agents, agents for maintaining a particular osmolality, chelators, proteins, lipids, small molecules, anticoagulants, etc. For example, in some preferred aspects of the present invention, a red blood cell sedimenting solution comprises physiological salt solutions, such as PBS, PBS lacking calcium and magnesium or Hank's balanced salt solution. In some preferred aspects of the present invention, heparin is present to red blood cell prevent clotting.

### Specific Binding Member for Removing Undesirable Components

In addition to the components of a sedimenting solution of the present invention, a combined solution of the present invention can comprise at least one specific binding member that can selectively bind undesirable components of a blood sample other than RBCs. One or more specific binding members that can selectively bind non-RBC undesirable components of a blood sample can be used to remove the undesirable components of the sample, increasing the relative proportion of rare cells in the sample, and thus contribute to the enrichment of rare cells of the sample.

A specific binding member that can specifically bind white blood cells can be as nonlimiting examples, an antibody, a ligand for a receptor, transporter, channel or other moiety of the surface of a white blood cell, or a lectin or other protein that can specifically bind particular carbohydrate moieties on the surface of a white blood cell (for example, a selectin).

Specific binding members that selectively bind to one or more undesirable components of the present invention can be used to capture one or more non-RBC undesirable components, such that one or more desirable components of the fluid sample can be removed from the area or vessel where the undesirable components are bound. In this way, the undesirable components can be separated from other components of the sample that include the rare cells to be separated. The capture can be affected by attaching the specific binding members that recognize the undesirable component or components to a solid support, or by binding secondary specific binding members that recognize the specific binding members that bind the undesirable component or components, to a solid support, such that the undesirable components become attached to the solid support. In preferred embodiments of the present invention, specific binding members that selectively bind undesirable sample components provided in a combined solution of the present invention are coupled to a solid support, such as microparticles, but this is not a requirement of the present invention.

Sedimenting solutions can be made and tested for their ability to sediment white blood cells and allow target cells to be recovered from the supernatant by adding the solutions to blood cells, mixing the blood sample and sedimenting solution, and incubating the blood sample for a period of time, after which the supernatant (unsedimented portion) is examined for the presence and amount of white blood cells. The volume of sedimenting solution added to the blood sample and the time of incubation can be varied in the testing of a potential sedimenting solution. Because the present invention seeks to increase the efficiency of enriching target cells of a blood sample, incubation times of less than an hour are preferred. Preferably, a white blood cell sedimenting solution of the present invention removes at least 50% of the white blood cells of a sample, more preferably, at least 90% of the white blood cells of a sample, and more preferably yet, at least 99% of the white blood cells of a sample after a mixing period of 30-60 minutes followed by a settling time of 30-60 minutes

E. Method of Enriching Rare Cells in a Blood Sample Using a Solution that Selectively Depletes Red and White Blood Cells

The present invention also includes a method of enriching rare cells of a blood sample using a solution that selectively sediments red blood cells and white blood cells. The method includes: adding a red blood cell sedimenting solution and white blood cell sedimenting solution of the present invention to a sample such as blood, mixing the sample and the sedimenting solutions, allowing red blood cells and white blood cells to sediment from the sample, and removing a supernatant that comprises enriched rare cells.

### Blood Sample

A blood sample can be any blood sample, recently taken from a subject, taken from storage, or removed from a source external to a subject, such as clothing, upholstery, tools, weapons, etc. A blood sample can therefore be an extract obtained, for example, by soaking an article containing blood in a buffer or solution. A blood sample can be unprocessed or partially processed, for example, a blood sample that has been dialyzed, had reagents added to it, etc. In some cases, it can be preferably to use a washed blood sample, in which blood cells have been pelleted and resuspended in a blood-compatible buffer (for example, PBE) at least once. A blood sample can be of any volume. For example, a blood sample can be less than five microliters, or more than 5 liters, depending on the application. Preferably, however, a blood sample that is processed using the methods of the present invention will be from about 10 microliters to about 2 liters in volume, more preferably from about one milliliter to about 250 milliliters in volume, and most preferably between about 5 and 50 milliliters in volume.

### Addition of RBC and WBC Combined Solution to Sample

A red and white blood cell sedimenting solution can be added to a blood sample by any convenient means, such as pipeting, automatic liquid uptake/dispensing devices or systems, pumping through conduits, etc. In most cases, the blood sample will be in a tube that provides for optimal separation of sedimented cells, but it can be in any type of vessel for holding a liquid sample, such as a plate, dish, well, or chamber. The amount of sedimenting solution that is added to a blood sample can vary, and will largely be determined by the concentration of red blood cell aggregation and linking solution and white blood cell aggregation and linking solution (as well as other components), so that their concentrations will be optimal when mixed with the blood sample.

Optimally, the volume of a blood sample is assessed, and an appropriate proportional volume of sedimenting solution, ranging from 0.01 to 100 times the sample volume, preferably ranging from 0.1 times to 10 times the sample volume, and more preferably from 0.25 to 5 times the sample volume, and even more preferably from 0.5 times to 2 times the sample volume, is added to the blood sample. (It is also possible to add a blood sample, or a portion thereof, to a red blood cell sedimenting solution. In this case, a known volume of sedimenting solution can be provided in a tube or other vessel, and a measured volume of a blood sample can be added to the sedimenting solution.)

### Mixing

The blood sample, red blood cell sedimenting solution, and white blood cell sedimenting solution are mixed so that the aggregating agent and one or more specific binding members of the sedimenting solution, as well as the components of the blood sample are distributed throughout the sample vessel. Mixing can be achieved means such as electrically powered acoustic mixing, stirring, rocking, inversion, agitation, etc., with methods such as rocking and inversion, that are least likely to disrupt cells, being favored. Mixing can be performed at any temperature from about 5 degrees C. to about 37 degrees C. In most cases, it is convenient to perform the steps of the method from about 15 degrees C. to about 27 degrees C. The optimal time for the mixing incubation can be determined empirically for a given mixing after adding the sedimenting solutions, varying the concentrations of the reagents, and the temperature of incubation. Preferably, the mixing incubation is from five minutes to twenty four hours in length, more preferably from ten minutes to ten hours in length, and most preferably from about fifteen minutes to about five hour in length. In some preferred aspects of the present invention, the incubation period is about thirty to sixty minutes

### Incubation of Blood Sample and Sedimenting Solution

The sample mixed with sedimenting solution is allowed to incubate to allow red blood cells to sediment. Preferably the vessel comprising the sample is stationary during the sedimentation period so that the cells can settle efficiently. Sedimentation can be performed at any temperature from about 5 degrees C. to about 37 degrees C. In most cases, it is convenient to perform the steps of the method from about 15 degrees C. to about 27 degrees C. The optimal time for the sedimentation incubation can be determined empirically for a given sedimenting solution after adding the sedimenting solutions, varying the concentrations of the reagents, and the temperature of incubation. Preferably, the sedimentation incubation is from five minutes to twenty four hours in length, more preferably from ten minutes to ten hours in length, and most preferably from about fifteen minutes to about five hour in length. In some preferred aspects of the present invention, the incubation period is about thirty to sixty minutes.

### Collecting Enriched Cells

Removing a supernatant (or a portion thereof) from the sample after the red blood cells have sedimented can be performed by pouring, pipeting, pumping, or a fluid uptake device. The supernatant comprises enriched rare cells of the blood sample, such as, but not limited to, stem cells, fetal cells, nucleated red blood cells, subpopulations of blood cells (e.g. T cells, B cells, NK cells, monocytes and mononuclear cells, and polymorphonuclear cells), non-hematopoietic cells (e.g. epithelial cells), cancer cells, virus-infected cells, parasite-infected cells, parasitic cells, or bacterial cells. Following RBC and WBC sedimentation with the sedimenting solutions of the present invention, the proportion of the rare cells to the other cell types in the sample has increased, thus resulting in enriched rare cells.

### F. Fetal Cell Identification

There are different types of fetal cells that can enter the maternal blood stream. They are: fetal nucleated red blood cells, fetal stem cells, fetal trophoblasts, fetal granulocytes and fetal lymphocytes.

There are some adult cells that express fetal hemoglobin and adult hemoglobin (called F-cells) which ranges from 0.3 to 4.4% of the erythrocytes. The ratio of gamma hemoglobin to beta hemoglobin in adult cells would have lower fetal hemoglobin compared to adult hemoglobin (up to 25% fetal to beta hemoglobin) (Rochette J, Craig J E, Thein S L. Fetal hemoglobin levels in adults. Blood Rev. December 1994; 8(4):213-24.). Therefore, it is possible to use adult beta hemoglobin as a differential expression level to remove the adult cells that express fetal hemoglobin. By using two colors, the ratio will distinguish the fetal from the maternal cell.

Fetal trophoblasts can be detected using trophoblast antigens such as beta human chorionic gonadotropin, placental alkaline phosphatase, (Yamanishi D T, Xu J, Hujsak P G, Yang Z, Wang X B, and Wu L. Expert Rev Mol Diagn.; 2(4):303-11, 2002).

### G. Cancer Cells

It is possible for solid malignant tumors to have the potential to enter the circulatory system, which are called circulating tumor cells. The cells have the potential to form metastases, autonomous secondary tumors, in other organs in the body. The spread of these malignant cells can determine the patient's prognosis.

The requirements of tumor staging or monitoring are-to diagnose the primary tumor or evaluate the secondary lesions early before they clinically manifest. The detection of circulating tumor cells would make it possible to initiate a possible treatment at an earlier date before the clinical appearance of a secondary lesion.

Body fluids contain a complex mixture of blood cells and may contain other non-hematopoietic cells. It is possible to enrich tumor cells either positive enriching for the tumor cells or depleting the non-tumor cells.

Body fluids can be but are not limited to blood, bone marrow aspirates, pleural effusion, lymph nodes extracts, and urine.

### H. Qualification of DNA Sequences

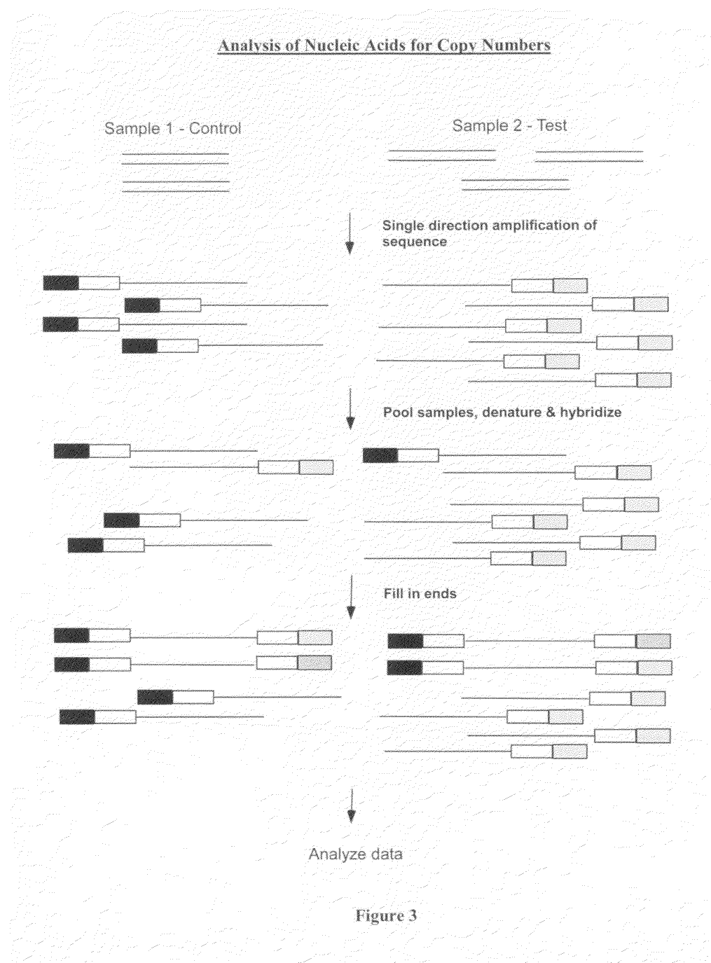

Cells may be mixture of target and background cells. The cells could be from a processed sample. In order to determine chromosome copy number, the cells will be lysed and DNA isolated. As shown in FIG. 3, genomic DNA was isolated. Primer sets were hybridized to Control or Test for a single direction amplification (either anti-sense or sense direction). This resulted in a linear amplification of specific region of DNA. This amplified sequence could be multiple sequences within a larger region or multiple regions in a specific chromosome or chromosomes.

The amplified DNA is recovered, concentrated and resuspended in hybridization buffer. This mixture was denatured and allowed to hybridize. After hybridization, this mixture was filled in using a DNA synthesis reaction. This result in double-stranded DNA (one copy of Control hybridized with one copy of Test) and single stranded DNA (either Control or Test ssDNA). The primers have a subregion that has a high denaturing temperature as part of the entire primer. This would result in the amplified double-stranded DNA that requires extensive denaturing to become single-stranded.

A second method is to include a non-homologous DNA sequence to be spiked in the Test and Control samples. This will be spiked into each sample before the single direction amplification. This non-homologous sequence would be used as a reference control for the reaction. The spike is used as a 1:1 ratio or other ratios. This is done to control for differences in the protocol and can be used as a reference that other reactions can be compared to. The ratio of the two experiments is used as a differential comparison for estimating nucleic acid copy numbers.

There are several possibilities for subsequent analysis. One is using real-time PCR to amplify the single-stranded DNA. Another is using a CGH and microarray procedures to evaluate the difference in copy numbers for the single stranded DNA. A third is the digestion of single-stranded DNA containing labeled primers and an evaluation of fluorescent tags from labeled primers.

### EXAMPLES

### Example 1

### WBC Depletion Combined with RBC Lysis to Enrich Target Cells

### Antibody Mixture

One aspect is to use an antibody coated non-magnetic bead to bind to a WBC specific antigen but which is not utilized to induce WBC aggregation or activation. One way to do this is to use an antibody to WBCs (e.g. CD50) that would specifically bind WBCs.

To improve WBC depletion, another reagent is utilized to activate cell:cell interactions. Antibodies can be used to induce WBCs to either bind specifically to other (homotypic, e.g. T cell to T cell) or bind specifically to another cell type (heterotypic, e.g. T cell to B cell). One example is using antibodies to CD98 (e.g. mAb 80A10) and CD3 (e.g. mAb OKT3) to mediate human T cell co-activation and induce homotypic aggregation (Miyamoto Y J, Mitechell J S, and McIntyre B W Mol Immunol 39: 739-751, 2003 and Bednarczyk J L and McIntyre B W J Immunol 144: 777-784, 1990).

### RBC Lysis of Blood

Peripheral blood is treated with 4 times volume of RBC lysis solution (e.g. 0.8% ammonium chloride solution with 0.1 mM EDTA, StemCell Technologies, Vancouver, Canada) at room temperature to lyse RBCs. Mixture is centrifuged at 200×g for 10 minutes to pellet cells and the supernatant was removed.

Antibody Depletion of White Blood Cells from Sample

Cell pellet is resuspended by gently tapping on tube. Cells are resuspended in PBE (PBS with 5% BSA and 5 mM EDTA), antibody to CD50 coated beads and antibodies added to the cell suspension, and mixed for 30-60 minutes. Cell suspension is allowed to settle for 30-60 minutes. Two layers will form: one is the aggregated cells and the other is the supernatant. Cells in supernatant are recovered from settled cell aggregates. Cells in supernatant is pelleted and counted.

### Example 2

### WBC and RBC Depletion

### Information on Cell Aggregation Solution

This protocol uses a solution containing three aspects. One aspect is a solution to induce RBC aggregation. This aspect is done using a dextran solution to chemically induce RBC aggregation combined with an antibody to glycophorin A (GpA).

The second aspect is a solution to link WBCs. A bead coated with an antibody to a leukocyte antigen (e.g. CD50, list additional examples) to partially link WBCs to WBCs can be used with the amount of beads ranging from 1 to 40 beads per WBCs.

The third aspect is a solution to aggregate WBCs. There are known antibodies that can induce either heterotypic (cell type to different cell type like T-cell to B-cell) or homotypic (similar cell type to similar cell type, like T-cell to T-cell) aggregation. These antibodies can include but are not limited to the following:
mAb G28-5 to CDw40 binds to lymphocytes for heterotypic cell aggregation (Gordon J et al J Imunnology 140: 1425-1430, 1988).
MAb JS64 to CD81 binds to lymphocytes and lymphocyte precursors (Lagaudriere-Gesbert C et al Cell Immunology 182, 105-112, 1997).

MAb BS-1 (best), B1-B6, WR14, G28-10, and G19-1 to CD43 can induce lymphocyte aggregation (Kuijpers T W et al J Immunol 149: 998-1003, 1992).
MAb GoH3 to VLA-6alpha (CD49f, A Sonnenberg Amsterdam, Netherlands) can induce lymphocytes to aggregate (Wuthrich R P Immunology 77: 214-218, 1992)
MAb HP1/1, HP1/3, HP1/7, HP2/7, HP2/4 (best) to VLA-4 (CD49d) induces lymphocyte aggregation (Campanero M R et al J Cell Biol 110: 2157-2165, 1990).
MAb L25 to VLA-4 (CD49d) induces lymphocyte aggregation (Bednarczyk J L et al J of Immunol 144: 777-784, 1990).
MAb 20E4 and 19H8 to VLA4 (CD49d) induce lymphocyte aggregation (Munn L et al J Immunol Methods 166: 11-25, 1993).
MAb B12 (N Ling University of Birmingham UK) to CD19 can induce B cells to aggregate and enhance with PMA (Smith S H et al Immunology 73: 293-297, 1991).
MAb CAL3.10 to CD50 can induce polymorphonuclear cells aggregation (Feldhaus M J et al J Immunology 161: 6280-6287, 1998).
   Preparation and Antibody Depletion of Cells from Sample

Blood is washed twice with salt solution to remove platelets and serum proteins. The wash solution is PBE (PBS with 0.5% BSA and 5 mM EDTA) and added to fill the tube. The sample is centrifuged at ˜200×g for 10 minutes and the solution removed from cell pellet. The washed sample is incubated with a RBC depleting solution (2% dextran solution with 1 ug of antibody to GpA per 10 mls of solution) and WBCs depleting solution (antibody coated beads with antibody to induce a subset of WBCs to aggregate). The depleting solution is added to washed blood in equal amounts for 30-60 min with gentle rotation. The cells are allowed to settle for 30-60 minutes. The supernatant is separated from aggregated cells. The cells are pelleted by centrifugation. The enriched cells are analyzed.

### Example 3

### Using Chemicals or Lectins to Deplete WBCs with RBC Aggregation

WBCs can be induced to aggregate using chemicals or lectins. Known reagents include but are not limited to the following:
Endothelin-1 (21 amino acid peptide) induces homotypic aggregation in granulocytes (Jozsef 1 et al. Brit J of Pharm 135:1167-1174, 2002)
PMA (1 uM) to induce granulocytes to aggregate (Draskovic-Pavlovic Immunology 96: 83-89, 1999)
Formyl peptide (1 μM; CHO-Nle-Leu-Phe-Nle-Tyr-Lys) will induce neutrophils to aggregrate (Simon S I et al J of Immunology 149: 2765-2771, 1992)
Bifenthrin (10-4 M) induces T cell aggregation (Hoffinan N et al Med Sci Monit 12: BR87-94, 2006)
Asialofetuin can induce tumor cells to homotypic aggregate (Meromsky L et al Cancer res 46: 5270-5275, 1986).
   Preparation and Antibody Depletion of Cells from Sample

Blood is washed twice with salt solution to remove platelets and serum proteins. The wash solution is PBE (PBS with 0.5% BSA and 5 mM EDTA) and added to fill the tube. The sample is centrifuged at ˜200×g for 10 minutes and the solution removed from cell pellet. The washed sample is incubated with a RBC depleting solution (2% dextran solution with 1 ug of antibody to GpA per 10 mls of solution) and WBCs depleting solution (antibody coated beads with either a chemical or lectin to induce a subset of WBCs to aggregate). The depleting solution is added to washed blood in equal amounts for 30-60 min with gentle rotation. The cells are allowed to settle for 30-60 minutes. The supernatant is separated from aggregated cells. The cells are pelleted by centrifugation. The enriched cells are analyzed.

### Example 4

### Double Separation

Another aspect to the method is to add a second step to further purify the target cells. The first step follows the examples presented above. Then the second enrichment step includes a magnetic step (either depletion or enrichment) or biochip (e.g. filter chip to deplete red or white blood cells).

Examples of a magnetic step uses a MACS (Miltenyi Biotec, Auburn, Calif.) microbead coated with an antibody to CD71, transferrin receptor, (or another cell surface antigen) to enrich nucleated RBCs and WBCs and remove some RBCs (Yamanishi, D T et al, Expert Reviews Mol Diagn 2(4): 303-311, 2002). Another example utilizes a biochip to deplete RBCs and retain the target cells (Mahamed H et al J Chromatogr A early publication, 2007)

### Example 5

### Identification of Fetal Cells

Beta versus Fetal and Embryonic Hemoglobin Antibody Staining

### Background Information

Fetal cells can be identified using labeled antibodies to two different surface markers. One example would be using fluorescent labeled antibodies and comparing the ratio of these fluorescent labels. Cells from the fetus are identified using antibodies to embryonic hemoglobin (epsilon hemoglobin) and fetal hemoglobin (gamma hemoglobin). Since there are adult cells that express gamma hemoglobin, it is possible to label adult cells with an antibody to gamma hemoglobin. In order to detect only fetal cells rather than a combination of fetal and adult cells, it is possible to use a ratio of labeled antibodies to gamma and beta hemoglobin to differentiate fetal versus adult cells. The method preferably requires at least 3-fold increase in labeled cells to be identified as fetal. This is possible using two fluorescent labels, green (e.g. FITC) versus red (e.g. Texas Red), to identify fetal cells. An antibody to gamma hemoglobin is labeled with a green fluorescent molecule and an antibody to beta hemoglobin is labeled with a red fluorescent molecule.

### Method

One hundred microliter aliquots of suspended separated cells are loaded onto slides precoated with 50 microliters of PBE. The slides are centrifuged at 600 rpm for 2 minutes, and then the slides are air dried for one to two minutes. The slides are fixed in Streck Tissue Fixative for 10 minutes, post-fixed in 2% formaldehyde/Streck for 4 minutes, and then washed in distilled water for a few seconds, in PBS twice for 6 minutes, in distilled water for five minutes, and then dried at 37 degrees C. The slides are used immediately or stored at −20 degrees C.

The slides are warmed to room temperature, when necessary, for 30-60 minutes, and then cell spots are isolated using a PAP-PEN (minimum size). The slide is blocked with 10% normal mouse serum/TBST (TBST, 50 mM Tris-HCl in 150 mM NaCl with 0.2% Tween 20) for 30 minutes at room temperature, and then incubated with fifty microliters of diluted antibodies (mouse anti-hemoglobin beta Texas Red, mouse anti-hemoglobin gamma-fluorescein, and mouse anti-hemoglobin epsilon fluorescein) at room temperature for 30 minutes. The slides are washed 4 times with TBST (5 minutes each with or without gentle shaking).

### Example 6

### Identification of Fetal Cells

Beta Versus Fetal and Embryonic Hemoglobin Staining with X/Y Chromosome Probes

Fetal cells can be identified using fluorescent labeled antibodies to two different surface markers. Cells from the fetus are identified using antibodies to embryonic hemoglobin (epsilon hemoglobin) and fetal hemoglobin (gamma hemoglobin). Since there are adult cells that express gamma hemoglobin, it is possible to label adult cells with an antibody to gamma hemoglobin. In order to detect only fetal cells rather than a combination of fetal and adult cells, it is possible to use a ratio of labeled antibodies to gamma and beta hemoglobin to differentiate fetal versus adult cells. The method preferably requires at least a 3-fold increase in labeled cells to be identified as fetal. This is possible using antibodies bound to two fluorescent colors, green versus red, to identify fetal cells. An antibody to gamma hemoglobin is labeled with a green fluorescent molecule and an antibody to beta hemoglobin is labeled with a red fluorescent molecule.

### Method

One hundred microliter aliquots of suspended separated cells are loaded onto slides precoated with 50 microliters of PBE. The slides are centrifuged at 600 rpm for 2 minutes, and then the slides are air dried for one to two minutes. The slides are fixed using formaldehyde, and then washed in distilled water for a few seconds, in PBS twice for 6 minutes, in distilled water for five minutes, and then dried at 37 degrees C. The slides are used immediately or stored at −20 degrees C.

If the slides are stored frozen, the slides are warmed to room temperature, when necessary, for 30-60 minutes, and then cell spots were isolated using a PAP-PEN (minimum size). The slides are blocked with 10% normal mouse serum/TBST for 30 minutes at room temperature, and then incubated with fifty microliters of diluted antibodies (mouse anti-hemoglobin beta Texas Red, mouse anti-hemoglobin gamma-fluorescein, and mouse anti-hemoglobin epsilon fluorescein) at room temperature for 30 minutes. The slides are washed 4 times with TBST (5 minutes each with or without gentle shaking).

In some cases, hemoglobin staining is checked prior to proceeding with FISH. The slides are air dried and mounted with 50% Glycerol/PBS and a coverslip. Then the coverslip is flipped off and the slide was rinsed twice in TBST for 5 minutes each and twice in distilled water for 1 minute. The slide is dehydrated in 70%, 95% and 100% ethanol for 2 minutes each and air dried.

If needed, cell spots on the slides are re-isolated with the PAP-PEN (minimum size). Ten microliters of a mixture of X and Y chromosome probes are added onto each cell spot, and the spots are covered with coverslips. A mixture of X and Y probe are added and hybridization is performed using methods known in the art.

The coverslips are gently removed. The slides are then washed in 2.times.SSC (saline sodium citrate) for 5 minutes. The slides are incubated with 1 microgram of Hoechst 33342 per ml of PBE for 5 min in the dark. The slides are rinsed in TBST for 5 minutes, then rinsed in distilled water twice for 1 minute each and air dried. The slides are mounted with Vectashield mounting medium and sealed with nail polish.

The cells are analyzed using a microscope to detect the two colors and compare the ratio of the intensity of the two fluorescent molecules.

### Example 7

### Identification of Fetal Cells

### Trophoblast and Fetal Cell Antibody Staining

Fetal cells can be identified using two fluorescent labeled antibodies to two different surface markers. Cells from the fetus are identified using antibodies to embryonic hemoglobin (epsilon hemoglobin) and fetal hemoglobin (gamma hemoglobin). Since there are adult cells that express gamma hemoglobin, it is possible to label adult cells with an antibody to gamma hemoglobin. In order to detect only fetal cells rather than a combination of fetal and adult cells, it is possible to use a ratio of the intensity of the labeled antibodies to gamma and beta hemoglobin to differentiate fetal versus adult cells. The method preferably requires at least 3-fold increase in labeled cells to be identified as fetal. This is possible using a ratio in the intensity of the two fluorescent molecules, green versus red, to identify fetal cells. An antibody to gamma hemoglobin is labeled with a green molecule and an antibody to beta hemoglobin is labeled with a red molecule.

Trophoblast cells can be identified using an antibody cocktail. Trophoblast cells express trophoblast and epithelial markers. An antibody cocktail of antibodies to epithelial membrane antigen, 5T4, LK26, plasminogen activator inhibitor 1, epidermal growth factor receptor, and c-erbB2 will detect either epithelial cells or fetal trophoblast cells. Contamination of skin epithelial cells can be minimized by avoiding the first tube of a blood draw.

### Method

100 microliter aliquots of suspended separated cells are loaded onto slides precoated with 50 microliters of PBE. The slides are centrifuged at 600 rpm for 2 minutes, and then the slides are air dried for one to two minutes. The slides are fixed in Streck Tissue Fixative for 10 minutes, post-fixed in 2% formaldehyde/Streck for 4 minutes, and then washed in distilled water for a few seconds, in PBS twice for 6 minutes, in distilled water for five minutes, and then dried at 37 degrees C. The slides are used immediately or stored at −20 degrees C.

If stored frozen, the slides are warmed to room temperature before use, for 30-60 minutes, and then cell spots are isolated using a PAP-PEN (minimum size). The slide is blocked with 10% normal mouse serum/TBST for 30 minutes at room temperature, and then incubated with fifty microliters of diluted antibodies at room temperature for 30 minutes. The antibody cocktail can detect fetal cells (mouse anti-hemoglobin beta Texas Red, mouse anti-hemoglobin gamma-fluorescein, and mouse anti-hemoglobin epsilon fluorescein) or fetal trophoblast cells (mouse anti-epithelial membrane antigen, anti-5T4, anti-LK26, anti-plasminogen activator inhibitor 1, anti-epidermal growth factor receptor, and anti-c-erbB2). The slides are washed 4 times with TBST (5 minutes each with or without gentle shaking).

### Example 8

### Evaluate Copy Number of DNA Sequences

Cells can be a mixture of target and background cells. Cells can be enriched from original sample and ready for analysis.

The cells can be labeled with antibodies or nucleic acid probes to identify cells of interest. The cells can be laser manipulated into a tube or destroyed. Such technologies already exists (e.g. Acturus, P.A.L.M. microlaser Tech, Cyntellect, Inc) to either capture or destroy cells on physical surfaces, in suspension or in an environment. This can result in an enriched subpopulation for analysis.

In order to determine chromosome copy number, the cells of interest can be lysed and DNA isolated. The procedure is shown in FIG. 3 and briefly described as follows. DNA is linear amplified using one direction (e.g. anti-sense) for Test and another direction (e.g. sense) for the Control. A primer is used that has a high melting temperature, which will minimize double stranded DNA to be denatured to single stranded DNA. DNAs are recovered, concentrated, pooled, denatured, and hybridized. The hybridized DNAs are then treated with an enzyme solution to fill-in the double stranded DNAs as shown in FIG. 3. The solution has double stranded DNAs and single stranded DNAs. The double stranded DNAs are unable to denature, so single stranded DNAs are the available DNAs for analysis.

As an example, assume the Test has a ratio of target to background of 1 to every 9 cells.

The Test has ten times N (10N) cells (one N target cell could have three copies of chromosome 13 and the rest of the cells (nine N cells) could have the normal 2 copies). For the Control, it has cells with 2 copies of chromosome 13 for a similar total number (N) of cells.

After amplification of each for 20 cycles, the Control has 2 copies of chromosome 13, amplified 20 cycles of two copies of chromosome 13 from 10N cells for a total of 400N copies of a DNA sequence from chromosome 13. The Test has 9N cells with 2 copies of chromosome 13 and 1N cell with 3 copies of chromosome 13, amplified 20 cycles for a total of 420N copies (e.g. 360N+60N) of a DNA sequence from chromosome 13. Assuming 90% hybridization efficiency between Control and Test, this will result in a decrease of 360 copies of the DNA sequence. For the control, this will result in 360 copies of double-stranded DNA and 40 copies of single-stranded DNA. For the Test, this will result in 360 copies of double-stranded DNA and 60 copies of single-stranded DNA. The end result is a comparison of 40 ssDNA for Control versus 60 ssDNA for Test and a 1.5-fold difference between the two, which can be distinguished using current technology. This can be repeated for multiple DNA sequences of chromosome 13 for reproducibility and improve sensitivity and specificity for an accurate evaluation in chromosome copy numbers.

### Example 9

### Evaluate Copy Number of DNA Sequences

Cells can be a mixture of target and background cells. Cells can be enriched from original sample and ready for analysis.

The cells can be labeled with antibodies or nucleic acid probes to identify cells of interest. The cells can be laser manipulated into a tube or destroyed. Such technologies already exists (e.g. Acturus, P.A.L.M. microlaser Tech, Cyntellect, Inc) to either capture or destroy cells on physical surfaces, in suspension or in an environment. This can result in an enriched subpopulation for analysis.

In order to determine chromosome copy number, the cells of interest can be lysed and DNA isolated. The procedure is shown in FIG. 3 and briefly described as follows. DNA is linear amplified using one direction (e.g. anti-sense) for Test and another direction (e.g. sense) for the Control. A primer is used that has a high melting temperature, which will minimize double stranded DNA to be denatured to single stranded DNA. DNAs are recovered, concentrated, pooled, denatured, and hybridized. The hybridized DNAs are then treated with an enzyme solution to fill-in the double stranded DNAs as shown in FIG. 3. The solution has double stranded DNAs and single stranded DNAs. The double stranded DNAs are unable to denature, so single stranded DNAs are the available DNAs for analysis.

As an example, assume the Test has a ratio of target to background of 1 to every 4 cells. The Test has 1000 cells (200 target cell could have three copies of chromosome 13 and 800 cells could have the normal 2 copies of chromosome 13). For the Control, it has cells with 2 copies of chromosome 13 for a total of 1000 cells.

After amplification of each for 20 cycles, the Control has 2 copies of chromosome 13, amplified 20 cycles of two copies of chromosome 13 from 1000 cells for a total of 40,000 copies of a DNA sequence from chromosome 13. The Test has 800 cells with 2 copies of chromosome 13 and 200 cells with 3 copies of chromosome 13, amplified 20 cycles for a total of 48,000 copies (e.g. 36,000+12,000) of a DNA sequence from chromosome 13. Assuming 90% hybridization efficiency between Control and Test, this will result in a decrease of 36,000 copies of the DNA sequence. For the control, this will result in 36,000 copies of double-stranded DNA and 4,000 copies of single-stranded DNA. For the Test, this will result in 36,000 copies of double-stranded DNA and 12,000 copies of single-stranded DNA. The end result is a comparison of 4,000 ssDNA for Control versus 12,000 ssDNA for Test and a 3-fold difference between the two, which can be distinguished using current technology. This can be repeated for multiple DNA sequences of chromosome 13 for reproducibility and improve sensitivity and specificity for an accurate evaluation in chromosome copy numbers.

There are several possibilities for analysis. One possibility is using real-time PCR to amplify the single-stranded DNA. Since different DNA sequences can be used to synthesize the Test and Control sequences, it is possible to compare the two sequences (Test versus Control) using different primers. This allows the user to evaluate the remaining single stranded DNA to determine copy number for each Test and Control sample and result in a ratio for a numerical copy number comparison.

Another possibility is using labeled primers using the linear amplification step, which would result in labeled single stranded DNA. At the end of the method, there will exist single stranded DNA and double stranded DNA (containing primers with high denaturing temperatures). It is possible to analyze the single stranded DNA using a microarray to compare copy numbers between Test and Control samples.

Another possibility is digesting the single stranded DNA and comparing the released labeled primer dyes between Test and Control

### Example 10

### Evaluate Copy Number of DNA Sequences with Internal Reference

Cells can be a mixture of target and background cells. Cells can be enriched from original sample and ready for analysis.

The cells can be labeled with antibodies or nucleic acid probes to identify cells of interest. The cells can be laser manipulated into a tube or destroyed. Such technologies already exists (e.g. Acturus, P.A.L.M. microlaser Tech, Cyntellect, Inc) to either capture or destroy cells on physical surfaces, in suspension or in an environment. This can result in an enriched subpopulation for analysis.

In order to determine chromosome copy number, the cells of interest can be lysed and DNA isolated. The procedure is shown in FIG. 3 and briefly described as follows.

DNA is linear amplified using one direction (e.g. anti-sense) for Test and another direction (e.g. sense) for the Control. A primer is used that has a high melting temperature, which will minimize double stranded DNA to be denatured to single stranded DNA.

DNAs are recovered, concentrated, pooled, denatured, and hybridized. The hybridized DNAs are then treated with an enzyme solution to fill-in the double stranded DNAs as shown in FIG. 3. The solution has double stranded DNAs and single stranded DNAs. The double stranded DNAs are unable to denature, so single stranded DNAs are the available DNAs for analysis.

As an example, assume the Test has a ratio of target to background of 1 to every 9 cells. The Test has ten times N (10N) cells (one N target cell could have three copies of chromosome 13 and the rest of the cells (nine N cells) could have the normal 2 copies). For the Control, it has cells with 2 copies of chromosome 13 for a similar total number (N) of cells.

After amplification of each for 20 cycles, the Control has 2 copies of chromosome 13, amplified 20 cycles of two copies of chromosome 13 from 10N cells for a total of 400N copies of a DNA sequence from chromosome 13. The Test has 9N cells with 2 copies of chromosome 13 and 1N cell with 3 copies of chromosome 13, amplified 20 cycles for a total of 420N copies (e.g. 360N+60N) of a DNA sequence from chromosome 13. Assuming 90% hybridization efficiency between Control and Test, this will result in a decrease of 360 copies of the DNA sequence. For the control, this will result in 360 copies of double-stranded DNA and 40 copies of single-stranded DNA. For the Test, this will result in 360 copies of double-stranded DNA and 60 copies of single-stranded DNA. The end result is a comparison of 40 ssDNA for Control versus 60 ssDNA for Test and a 1.5-fold difference between the two, which can be distinguished using current technology. This can be repeated for multiple DNA sequences of chromosome 13 for reproducibility and improve sensitivity and specificity for an accurate evaluation in chromosome copy numbers.

A DNA sequence with a known copy number can be used as a reference for the amplification and subsequent steps of the protocol. The copy number ratio can be used to evaluate the efficiency of the experiment. It can be used as a reference such that a ratio of 1 may suggest equal copy number between two DNA sequences versus a ratio of greater than 1.5 may suggest either non-equal copy number or something incorrectly done during the experiment. This reference DNA could also be used as a reference to correct for differences during the protocol.

### Example 11

### Comparison of Two Samples to Evaluate Copy Number with Internal Reference

Cells can be a mixture of target and background cells. Cells can be enriched from original sample and ready for analysis.

The cells can be labeled with antibodies or nucleic acid probes to identify cells of interest. The cells can be laser manipulated into a tube or destroyed. Such technologies already exists (e.g. Acturus, P.A.L.M. microlaser Tech, Cyntellect, Inc) to either capture or destroy cells on physical surfaces, in suspension or in an environment. This can result in an enriched subpopulation for analysis.

In order to compare the copy number of a specific nucleic acid sequence or sequences between two samples, the cells of interest can be lysed and nucleic acids isolated. The procedure is shown in FIG. 3 and briefly described as follows.

The two samples will be compared using the Target to Control and Control to Control. This will allow the investigator to evaluate a specific nucleic acid sequence or multiple nucleic acid sequences with the two tests.

If the study was on RNA, RNA from one or both samples could be in vitro transcribed to cDNA. RNA can be hybridize to amplified DNA or cDNA to amplified DNA or cDNA to cDNA or amplified cDNA to amplified cDNA.

If the study was on genomic DNA, the DNA is linear amplified using one direction (e.g. anti-sense) for Test and another direction (e.g. sense) for the Control.

A primer is used that has a high melting temperature, which will minimize double stranded nucleic acids to be denatured to single stranded DNA.

The nucleic acids are recovered, concentrated, pooled, denatured, and hybridized. The hybridized nucleic acids are then treated with an enzyme solution to fill-in the double stranded DNAs as shown in FIG. 3. The solution has double stranded nucleic acids and single stranded nucleic acids. The double stranded nucleic acids are unable to denature, so single stranded nucleic acids are the available nucleic acids for analysis.

### Example 12

### Evaluate Copy Number of DNA Sequences with Spiked Reference DNA

Cells can be a mixture of target and background cells. Cells can be enriched from original sample and ready for analysis.

The cells can be labeled with antibodies or nucleic acid probes to identify cells of interest. The cells can be laser manipulated into a tube or destroyed. Such technologies already exists (e.g. Acturus, P.A.L.M. microlaser Tech, Cyntellect, Inc) to either capture or destroy cells on physical surfaces, in suspension or in an environment. This can result in an enriched subpopulation for analysis.

In order to determine chromosome copy number, the cells of interest can be lysed and DNA isolated. The procedure is shown in FIG. 3 and briefly described as follows. Before amplification, a non-homologous DNA sequence is spiked into the Control and Test samples. This non-homologous DNA will have no homology to either the Test or Control DNA and will be used to evaluate the amplification and subsequent steps.

DNA is linear amplified using one direction (e.g. anti-sense) for Test and another direction (e.g. sense) for the Control. A primer is used that has a high melting temperature, which will minimize double stranded DNA to be denatured to single stranded DNA.

DNAs are recovered, concentrated, pooled, denatured, and hybridized. The hybridized DNAs are then treated with an enzyme solution to fill-in the double stranded DNAs as shown in FIG. 3. The solution has double stranded DNAs and single stranded DNAs. The double stranded DNAs are unable to denature, so single stranded DNAs are the available DNAs for analysis.

As an example, assume the Test has a ratio of target to background of 1 to every 9 cells. The Test has ten times N (10N) cells (one N target cell could have three copies of chromosome 13 and the rest of the cells (nine N cells) could have the normal 2 copies). For the Control, it has cells with 2 copies of chromosome 13 for a similar total number (N) of cells.

After amplification of each for 20 cycles, the Control has 2 copies of chromosome 13, amplified 20 cycles of two copies of chromosome 13 from 10N cells for a total of 400N copies of a DNA sequence from chromosome 13. The Test has 9N cells with 2 copies of chromosome 13 and 1N cell with 3 copies of chromosome 13, amplified 20 cycles for a total of 420N copies (e.g. 360N+60N) of a DNA sequence from chromosome 13. Assuming 90% hybridization efficiency between Control and Test, this will result in a decrease of 360 copies of the DNA sequence. For the control, this will result in 360 copies of double-stranded DNA and 40 copies of single-stranded DNA. For the Test, this will result in 360 copies of double-stranded DNA and 60 copies of single-stranded DNA. The end result is a comparison of 40 ssDNA for Control versus 60 ssDNA for Test and a 1.5-fold difference between the two, which can be distinguished using current technology. This can be repeated for multiple DNA sequences of chromosome 13 for reproducibility and improve sensitivity and specificity for an accurate evaluation in chromosome copy numbers.

The non-homologous DNA can be used as a reference for the amplification and subsequent steps of the protocol. The copy number ratio can be used to evaluate the efficiency of the experiment. It can be used as a reference such that a ratio of 1 may suggest equal copy number between two DNA sequences versus a ratio of greater than 1.5 may suggest either non-equal copy number or something incorrectly done during the experiment. This non-homologous DNA could also be used as a reference to correct for differences during the protocol.

### Example 13

### Using Chemicals or Lectins to Deplete WBCs with RBC Aggregation

WBCs can be induced to aggregate using chemicals or lectins. Known reagents include but are not limited to the following:
Endothelin-1 (21 amino acid peptide) induces homotypic aggregation in granulocytes (Jozsef 1 et al. Brit J of Pharm 135:1167-1174, 2002)
PMA (1 uM) to induce granulocytes to aggregate (Draskovic-Pavlovic Immunology 96: 83-89, 1999)
Formyl peptide (1 μM; CHO-Nle-Leu-Phe-Nle-Tyr-Lys) will induce neutrophils to aggregrate (Simon S I et al J of Immunology 149: 2765-2771, 1992)
Bifenthrin (10-4 M) induces T cell aggregation (Hoffman N et al Med Sci Monit 12: BR87-94, 2006)
Asialofetuin can induce tumor cells to homotypic aggregate (Meromsky L et al Cancer res 46: 5270-5275, 1986).
   Preparation and Antibody Depletion of Cells from Sample

Blood is washed twice with salt solution to remove platelets and serum proteins. The wash solution is PBE (PBS with 0.5% BSA and 5 mM EDTA) and added to fill the tube. The sample is centrifuged at 200×g for 10 minutes and the solution removed from cell pellet. The washed sample is incubated with a RBC depleting solution (2% dextran solution with 1 ug of antibody to GpA per 10 mls of solution) and WBCs depleting solution (antibody coated beads with either a chemical or lectin to induce a subset of WBCs to aggregate). The depleting solution is added to washed blood in equal amounts for 30-60 min with gentle rotation. The cells are allowed to settle for 30-60 minutes. The supernatant is separated from aggregated cells. The cells are pelleted by centrifugation.

The cells are resuspended and then analyzed. It is anticipated that red blood cells are depleted 50% and white blood cells are depleted minimally at least 100, better at least 500 and best by at least 1000 fold.

### Example 14

### Example of Double Separation

The first step follows the examples presented above with the second enrichment step being a second separation, which either enriches the target cells or depletes the non-target or background cells. A possibility is using a magnetic step (either depletion or enrichment), a biochip (e.g. filter chip to deplete red or white blood cells) or a separation step to either enrich the target cells or deplete the non-target cells.

One example of a double separation procedure is the following. The steps of the enrichment procedure, going in sequential order: 1) washing the blood sample (2 centrifugations); 2) selectively sedimenting red blood cells and selectively removing white blood cells using a Depleting Reagent (PBS lacking calcium and magnesium containing: 5 millimolar EDTA, 2% dextran (molecular weight from 70 to 200 kilodaltons), 0.1 to 0.15 micrograms per milliliter of IgM antibodies to glycophorin A, an antibody to induce WBC aggregation (e.g. mAB B12), and approximately 1-5×10⁹ magnetic beads coated with a CD50 antibody and approximately 1-4×10⁹ magnetic beads coated with a CD31 antibody); and 3) separating the supernatant from step 2) of target cells through a microfabricated filter or cell sorter apparatus (e.g. FACS or cell purification system). The sample containing an enriched subpopulation of target cells could then be analyzed (including but not limited to deposition onto a microscope slide).

It is anticipated that the separation procedure described above would deplete at least 10-fold, better would be 100-fold, and best would be greater than or equal to 1000-fold of red blood cells. It is anticipated that the separation procedure described above would deplete at least 10-fold, better would be 25-fold, and best would be greater than or equal to 100-fold of white blood cells. It is anticipated that the separation procedure described above would at least recover one target cell from the sample and better would be two or more target cells from the sample.

The next step could be to identify cells. The cells on the slide could be fixed with a fixation agent (100% S.T.F. (Streck, Omaha, Nebr.) for 10 minutes and S.T.F.-0.75% PFA for 4 minutes), washed in water, and washed in 1×PBS for 6 minutes each. The cells could then be incubated with antibodies to the following antigens: including but not limited to anti-ε hemoglobin, anti-γ hemoglobin, anti-αc-fetoprotein, anti-Epidermal growth factor receptor (EGFR), and anti-c-erbB-2/HER2. Theses antibodies could be detected by primary labeling, secondary labeling, tertiary labeling, enzymatic labeling, chemical labeling or a combination of some or all types of labelings.

It is also possible to use probes to the RNA or DNA sequence to identify RNA or DNA present in fetal cells. The cells could then incubated with nucleic acids (including but not limited to e.g. oligonucleotides, antisense RNA or DNA oligonucleotides, peptide nucleic acids) to hybridize with the following sequences: including but not limited to anti-ε hemoglobin, anti-γ hemoglobin, anti-α-fetoprotein, anti-Epidermal growth factor receptor (EGFR), and anti-c-erbB-2/HER2. These sequences could be detected by primary labeling, secondary labeling, tertiary labeling, enzymatic labeling, chemical labeling or a combination of some or all types of labelings.

After the identification reaction (e.g. immunohistochemistry or immunocytochemical or in situ hybridization), the slides are washed and dehydrated with ethanol washes. This next step is to determine DNA copy number of chromosomes using fluorescent in situ hybridization. The slides are denatured by an incubated at 80 degrees Celsius for 1.75 to 5 minutes with labeled oligonucleotides (including but not limited to probes to chromosome X and Y, 0.5 μl of CEP X and 0.5 μL of CEP Y (Vysis, Downers Grove, Ill.), 2 μl of dH₂O, and 7 μl of CEP buffer solution). After melting the DNA, the slides are incubated at 37 degrees Celsius overnight. The slides are washed at 70 degrees Celsius for 20-30 seconds in 0.4×SSC and once in 2×SSC and 0.1% Igepal CA-630 for 1 minute. The nuclei are stained with a nuclear specific fluorescent dye. An anti-fading reagent (e.g. VectorShield Vector) is add to the cells and the slide is cover slipped. The cells on the slide could then be analyzed using fluorescent microscopy or other technologies.

From the identification procedures, we would suggest that some cells are labeled. From the steps described above, we would anticipate the target cells would be labeled as well as be able to determine the chromosome copy numbers for most cells. The potential fetal cells could be nucleated red blood cells (e.g. anti epsilon hemoglobin as a target antigen) or trophoblast cells (e.g. using an epidermal marker such as epidermal growth factor receptor or potential trophoblast marker such as alpha-fetoprotein as a target antigen) with the protein targets described above.

It is also possible to target the labeled cells for laser microdissection using fluorescent label(s) as a cell marker. The labeled cell(s) could be targeted for laser microdissection after cell enrichment, after cell labeling step or after interphase FISH procedure. The slide can be transferred to a laser capture device and catapult the desired cell onto a cap, which has been placed over the target area. Pulsing the laser through the cap causes the thermoplastic film to form a thin protrusion that bridges the gap between the cap and desired cell and adheres to the target cell. Lifting the cap removes the target cell(s) now attached to the cap. Biomolecules are extracted from the cells using DNA, RNA, or protein isolation kits.

By obtaining a relative pure subpopulation containing the target cells of interest (e.g. fetal cells), it is now possible to study the cells directly. This would result in the ability to study a further enriched fetal cell subpopulation using many technologies including but not limited to mass spec (e.g. protein and SNP analysis), microarray (e.g. chromosome alterations, point or genomic alterations and SNP analysis), and whole genome amplification for analysis of single to few cells (e.g. study point mutations or genomic alterations).

### Example 15

### Identification of Fetal Cells

### Beta Versus Fetal and Embryonic Hemoglobin Staining

One hundred microliter aliquots of suspended separated cells are loaded onto slides precoated with 50 microliters of PBE. The slides are centrifuged at 600 rpm for 2 minutes, and then the slides are air dried for one to two minutes. The slides are fixed using formaldehyde, and then washed in distilled water for a few seconds, in PBS twice for 6 minutes, in distilled water for five minutes, and then dried at 37 degrees C. The slides are used immediately or stored at −20 degrees C.

If the slides are stored frozen, the slides are warmed to room temperature, when necessary, for 30-60 minutes, and then cell spots were isolated using a PAP-PEN (minimum size). The slides are blocked with 10% normal mouse serum/TBST for 30 minutes at room temperature, and then incubated with fifty microliters of diluted antibodies (mouse anti-hemoglobin beta Texas Red, mouse anti-hemoglobin gamma-fluorescein, and mouse anti-hemoglobin epsilon fluorescein) at room temperature for 30 minutes. The slides are washed 4 times with TBST (5 minutes each with gentle shaking).

In some cases, hemoglobin staining is checked prior to proceeding with FISH. The slides are air dried and mounted with 50% Glycerol/PBS and a coverslip. Then the coverslip is flipped off and the slide was rinsed twice in TBST for 5 minutes each and twice in distilled water for 1 minute. The slide is dehydrated in 70%, 95% and 100% ethanol for 2 minutes each and air dried.

If needed, cell spots on the slides are re-isolated with the PAP-PEN (minimum size). Ten microliters of a mixture of X and Y chromosome probes are added onto each cell spot, and the spots are covered with coverslips. A mixture of X and Y probe are added and hybridization is performed using methods known in the art.

The coverslips are gently removed. The slides are then washed in 2.times.SSC (saline sodium citrate) for 5 minutes. The slides are incubated with 1 microgram of Hoechst 33342 per ml of PBE for 5 min in the dark. The slides are rinsed in TBST for 5 minutes, then rinsed in distilled water twice for 1 minute each and air dried. The slides are mounted with Vectashield mounting medium and sealed with nail polish.

The cells are analyzed using a microscope to detect the two colors and compare the ratio of the intensity of the two fluorescent molecules (ratio of red stained for adult and green stained for fetal). It is anticipated that for a cell to be labeled fetal, there is at least 3-fold higher ratio of green label (epsilon and gamma hemoglobin) to red label (beta hemoglobin). It is anticipated that for a cell to be labeled adult, there is less than 2-fold higher ratio of green label (epsilon and gamma hemoglobin) to red label (beta hemoglobin).

What is claimed is:

1. A method to enrich cells from body fluids comprising:
a) an agent to induce cell aggregation, and
b) an agent to link cells to cells.

2. The method of claim 1, wherein the agent to induce cell aggregation is an antibody, a protein, a growth factor, a lectin, a peptide, a carbohydrate, a lipid, or a chemical.

3. The method of claim 1, wherein the agent linking cells is an antibody, a linked antibody, an antibody linked to a surface, a protein linked to a surface, a lectin linked to a surface, a carbohydrate on a surface, a lipid on a surface, or a chemical linked to a surface.

4. A method for enriching and recovering cells from a biological sample
a) a composition for separating nucleated cells in a sample comprising:
i) at least one agent to induce nucleated cells to aggregrate;
ii) at least one agent to link nucleated cells to nucleated cells; and
iii) at least one agent to induce red blood cells to aggregate;
b) said composition being added to said body fluid and mixed; and
c) said mixture being allowed to partition into two layers, an aggregated and a supernatant phase.

5. The method of claim 4, wherein said agent to induce nucleated cells to aggregate is an antigen on white cells to induce the cell to aggregate is selected from the group consisting of CD3, CD2, CD40, CD81, CD43, CD49, CD94, CD161, CD15, CD72, CD8, and CD19.

6. The method of claim 4, wherein said agent to induce nucleated cells to aggregate is a formyl peptide, an endothelin-1 peptide, a bifenthrin, an asialofetuin, or a phorbol myristate acetate.

7. The method of claim 4, wherein said agent to link nucleated cells is linked to an antigen on nucleated cells.

8. The method of claim 7, wherein said antigen is at least one of the following: CD50, CD7, CD19, CD53, CD43, CD100, CD66 or CD45.

9. The method of claim 4, wherein the solution to induce red blood cells to aggregates comprises:
a) an antibody to an antigen on red blood cells, which is glycophorin A or
b) a solution to induce red blood cells to aggregate which comprises: dextran or hepastarch.

10. The method of claim 4, wherein said sample is a blood sample, an effusion, an urine sample, semen, fecal matter, cells in an aspirate or swab from the uterus, vaginal or nasal tissue, bone marrow aspirate, spinal fluid, fluid or wash from tissue or digested tissue, cell suspension from tissue, sputum, mucus, or saliva.

11. The method of claim 4, wherein said target cell is a fetal cell, cancer cell, neoplastic cell, tumor cell, stem cell, progenitor cell, or non-hematopoietic cell.

12. The method of claim 4, wherein said solution to induce red blood cells to aggregates comprises:
a) a protein or a lectin that will bind to an antigen on red blood cells, or
b) a solution to induce red blood cells to aggregate comprises: dextran or hepastarch.

13. The method of claim 4 further comprising a second enrichment step to enrich target cells from body fluids which comprises magnetic separation, filtration, density gradient, cell sorting, optical sorting, dielectrophoresis, RBC lysis, gel, plug, or second RBC aggregation step.

14. A method to identify target cells using ratio of two markers to differentiate target cells from background cells, wherein;
a) a marker is expressed higher in target cells compared to background cells;
b) a marker is expressed only in background cells; and
c) the ratio of the two markers is used to differentiate target cells from background cells.

15. The method of claim 14, wherein said markers are:
a) hemoglobin gamma or fetal hemoglobin,
b) hemoglobin epsilon or embryonic hemoglobin, or
c) hemoglobin beta or adult hemoglobin.

16. The method of claim 14, wherein said marker can consist of one or a combination of:
a) alpha fetoprotein,
b) epidermal growth factor receptor,
c) epithelial membrane antigen,
d) 5T4,
e) LK26,
f) plasminogen activator inhibitor 1, or
g) c-erbB-2/Her2/Neu.

17. A method to determine the nucleic acid copy number, comprising:
a) the nucleic acids from target cells and reference cells are amplified in a single direction,
b) the amplified nucleic acids are combined,
c) the combined sample is denatured and hybridized,
d) the double stranded nucleic acids are filled in, and
e) the single stranded nucleic acids are subsequently analyzed.
